(19) United States
(12) Patent Application Publication
(10) Pub. No.: US 2021/0158834 A1
(43) Pub. Date: May 27, 2021

(54) DIAGNOSING AND TREATMENT OF SPEECH PATHOLOGIES USING ANALYSIS BY SYNTHESIS TECHNOLOGY

(71) Applicant: Ninispeech Ltd., Haifa (IL)

(72) Inventors: Yoav MEDAN, Haifa (IL); Shai SHAPIRA, Ramat David (IL)

(21) Appl. No.: 17/046,777

(22) PCT Filed: Apr. 17, 2019
(86) PCT No.: PCT/IL2019/050442

§ 371 Date: Oct. 10, 2020

Related U.S. Application Data
(60) Provisional application No. 62/662,551, filed on Apr. 25, 2018.

(51) Int. Cl. G10L 21/057; G10L 17/04; G10L 17/26; G10L 15/06; G10L 13/033

(52) U.S. Cl. CPC G10L 21/057; G10L 17/04; G10L 2015/0631; G10L 15/063; G10L 13/033; G10L 17/26

(57) ABSTRACT

There are provided herein, a method and system for creating a speech/language pathologies classifier, the method comprising: producing a pathological speech repository of pathological speech samples of multiple impairments; computing speech qualities/pathologies, based on data receive from the pathological speech repository; producing a text repository, the text repository comprises multiple known text passages; converting each one of a selection of the text passages from the multiple known text passages, to a speech segment, while introducing to the speech segment one or more of the computed speech pathologies, thereby creating multiple synthetic impaired speech segments; and training a classifier with the multiple synthetic impaired speech segments thereby creating a speech/language pathologies classifier.

### FIELD OF THE INVENTION

Embodiments of the disclosure relate to speech/language pathologies.

### BACKGROUND

Traditionally, classification of speech pathologies for diagnosis and assessment of therapy progress are done subjectively by a trained human professional. More recently, computers have shown to be reliably capable of understanding human speech, using new approaches that rely on vast amount of tagged speech data (the text encoding and time alignment are known) and processing power. Such classification machines are various variants of what is called Deep Neural Networks (DNNs). Still, they fall short in classifying and understanding pathological speech and thus, are unable to diagnose and assess the quality of such speech.

There is a need in the art for improved and efficient methods and systems for diagnosing and treating speech/language related pathologies based on objective metrics.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

Initial attempts to bridge the gap between classification of normal speech and understanding pathological speech were based on analyzing the speech and applying a set of rules for detecting pathological events such as in stuttering. However, to improve the robustness of such classification machine and broaden its scope to other speech pathologies, such as, but not limited to, articulation, one would need large sets of high quality tagged pathological speech data, which do not currently exist and would cost a lot of resources to acquire.

There are thus provided, according to some embodiments, a method and system for generating unlimited amount of tagged speech training sets using synthetic pathological speech samples based on a known text and generated by a common Text-To-Speech (TTS) technology. According to some embodiments, the system (and method) include a module that is configured to “inject” typical speech pathologies into the generated speech, either at the text level and/or into the synthesized speech.

There are further provided, according to some embodiments, a method and system for providing a (fully) instrumented practice experience with objective Speech Quality (SQ) metrics and analytics. According to some embodiments, vocal prompting templates are based on the voice attributes, traits and/or qualities of a user (trainee), pitch range, loudness, timbre, pace, etc., such that it provides the user a vocal “mirror” (into the future), of his/her/their speech once the training/therapy ends successfully.

According to some embodiments, the attributes or traits of the user's voice are extracted by standard voice analysis approaches and may be embedded into a text-to-speech synthesis processor.

According to some embodiments, there is provided herein method of creating a speech/language pathologies classifier, the method comprising: producing a pathological speech repository of pathological speech samples of multiple impairments; computing speech qualities/pathologies, based on data receive from the pathological speech repository; producing a text repository, the text repository comprises multiple known text passages; converting each one of a selection of the text passages from the multiple known text passages, to a speech segment, while introducing to the speech segment one or more of the computed speech pathologies, thereby creating multiple synthetic impaired speech segments; and training a classifier with the multiple synthetic impaired speech segments thereby creating a speech/language pathologies classifier.

According to some embodiments, there is provided herein a method for personalized speech therapy, the method comprising: recording an actual speech sample provided by a user; and utilizing a speech/language pathologies classifier, computing one or more output signals indicative of one or more speech qualities of the user, wherein creating the speech/language pathologies classifier comprises: producing a pathological speech repository of pathological speech samples of multiple impairments; computing speech qualities/pathologies, based on data receive from the pathological speech repository; producing a text repository, the text repository comprises multiple known text passages; converting each one of a selection of the text passages from the multiple known text passages, to a speech segment, while introducing to the speech segment one or more of the computed speech pathologies, thereby creating multiple synthetic impaired speech segments; and training a classifier with the multiple synthetic impaired speech segments thereby creating a speech/language pathologies classifier.

According to some embodiments, training the classifier may include implementing a machine learning software. According to some embodiments, the output signal may further include one or more assigned speech quality scores.

According to some embodiments, the one or more speech qualities may include speech intelligibility, fluency, vocabulary, accent, emotion, pronunciation, jitter, shimmer, duration, intonation, tone, rhythm, or any combination thereof.

According to some embodiments, the output signal may further include one or more assigned speech intelligibility scores.

According to some embodiments, the method may further include providing a feedback signal to the user and/or to a caregiver.

According to some embodiments, producing the pathological speech repository of pathological speech samples of multiple impairments may include recording of speech samples from human subjects.

According to some embodiments, recording the actual speech sample may be provided by the user in response to a content-containing stimulus.

According to some embodiments, the content-containing stimulus may include a text section, a picture, an image, a video clip, a vocal section or any combination thereof, presented to the subject.

According to some embodiments, there is further provided herein a system of creating a speech/language pathologies classifier, the method comprising: a speech qualities module configured to compute speech qualities/pathologies based on data receive from a pathological speech repository of pathological speech samples of multiple impairments; a Text to Speech module configured to convert text passages, obtained from a text repository comprising multiple known text passages, to a speech segments, while introducing to the speech segments one or more of the computed speech pathologies, thereby creating multiple synthetic impaired speech segments; and a classifier configured to receive the multiple synthetic impaired speech segments thereby form a speech/language pathologies classifier.

According to some embodiments, there is further provided herein a system for personalized speech therapy, the system comprising: a recorder configured to record an actual speech sample of a user; and a processor comprising: a speech qualities module configured to compute speech qualities/pathologies based on data receive from a pathological speech repository of pathological speech samples of multiple impairments; a Text to Speech module configured to convert text passages, obtained from a text repository comprising multiple known text passages, to a speech segments, while introducing to the speech segments one or more of the computed speech pathologies, thereby creating multiple synthetic impaired speech segments; and a speech/language pathologies classifier configured to receive the multiple synthetic impaired speech segments and the recorded speech sample of the user and to produce an output signal indicative of one or more speech qualities of the user.

According to some embodiments, the system may further include a recorder configured to record a text sample of a user and to introduce it to the speech/language pathologies classifier. According to some embodiments, the system may further include a display configured to present the one or more speech qualities of the user. According to some embodiments, the system may further include a loudspeaker, configured to play back a modified speech to the user.

According to some embodiments, there is further provided herein a method of training a subject suffering from a speech pathology, the method comprising: recording a user's speech section; utilizing voice analysis algorithms, analyzing the user's speech section to identify at least one speech impairment; modifying the identified speech impairment to produce a synthetic speech section comprising a modified speech impairment; and playing to the user the synthetic speech section having the modified speech impairment, thereby providing a feedback to the user regarding the speech thereof.

According to some embodiments, the synthetic speech section may be produced by using or mimicking the user's own voice or one or more voice qualities of the user.

According to some embodiments, modifying the speech impairment may include removing the speech impairment. According to some embodiments, modifying the speech impairment may include adjusting the level of the speech impairment. According to some embodiments, modifying the speech impairment may include shifting the time and/or frequency of the impairment.

According to some embodiments, playing to the user the synthetic speech section may include playing the section in a time delay (Delayed Auditory Feedback).

According to some embodiments, the method may further include computing a speech quality score based on a comparison between the user's recorded speech and a template (normal) speech section.

According to some embodiments, there is further provided herein a method of producing synthetic impaired speech sections, the method comprising: providing recorded impaired speech sections of one or more users; selecting one or more speech impairments in each of the impaired speech sections; producing synthetic impaired speech sections by controllably manipulating (adjusting/modifying) the level of the one or more selected speech impairments; and tagging each of the synthetic impaired speech sections based on the type and severity of the speech impairment(s) thereof. According to some embodiments, the speech impairment may relate to a vocal articulations skill.

According to some embodiments, the tagging of each of the synthetic impaired speech sections may further be based on quantification of the impairment relative to prototype norms of normal speech.

According to some embodiments, the synthetic impaired speech sections may be searchable and anonymous.

More details and features of the current invention and its embodiments may be found in the description and the attached drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below:

FIG. 1 schematically depicts a block diagram of a system for treating/diagnosing a speech/language related pathology, according to some embodiments; and

FIG. 2 schematically depicts a flowchart of a method for treating/diagnosing a speech/language related pathology, according to some embodiments.

### DETAILED DESCRIPTION

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

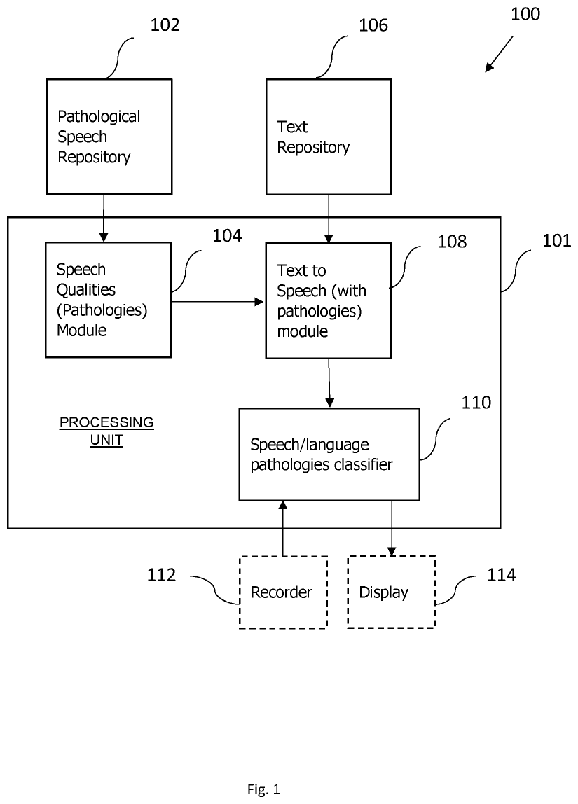

Reference is now made FIG. 1, which schematically depicts a block diagram of a system 100 for treating/diagnosing a speech/language related pathology, according to some embodiments. The system may also be used to provide analytics during and/or following therapy.

System 100 includes a pathological speech repository 102, a Speech Quality (SQ) module 104, a text repository 106, a Text to Speech (TTS) module 108, and a classifier 110.

Speech Quality (SQ) module 104, Text to Speech (TTS) module 108 and a classifier 110 may be separate modules or a part of a processing circuitry 101.

Pathological speech repository 102 is a collection of pathological speech samples recorded of different impairments (for example, but not limited to, stuttering, pronunciation pathologies, phonation pathologies, voice related pathologies, Parkinson related speech impairment, impaired articulation language impairments, etc.). According to some embodiments, the samples are recordings of pathological speech utterances, with tags/metadata indicating the time interval and type of each pathological speech segment.

Speech Quality module 104 is configured to receive data from pathological speech repository 102 and to compute speech qualities (SQs). Speech qualities may include, for example, parameters, features and/or attributes of speech impairments that will be needed to drive the Text-To-Speech (TTS) synthesis.

Text Repository 106 includes a collection of text passages. According to preferred embodiments, the text passages are known, to facilitate proper tagging of the resulting speech. The text passages may include passages used in standard tests and/or treatment protocols, for example, “Rainbow Passage” commonly used for Parkinson. More details of such protocols may be found in: http://citeseerx.ist.psu.edu/viewdoc/download?doi=10.1.1.1006.9218&rep=rep1&type=pdf, which is incorporated herein by reference in its entirety.

Text to Speech (TTS) module 108, is configured to convert the text passages (from text repository 106) to speech, while introducing to the produced speech the speech pathologies computed by Speech Quality module 104, thus creating multiple synthetic impaired speech segments. The synthetic impaired speech segments created by TTS module 108 are used to train classifier 110, thus creating a speech/language pathologies classifier. Classifier 110, which is now trained as a speech/language pathologies classifier may implement machine learning software, such as, but not limited to, Deep Neural Networks (DNN), decision trees, and statistical models.

System 100 may further include a recorder 112 configured to record a speech (spoken text) sample of a user and to introduce it to the user's speech/language pathologies classifier. Recording the speech (spoken text) sample of the user and introducing it to the speech/language pathologies classifier 110, will provide an output indicative of the user's speech qualities.

System 100 may further include a display 114 configured to present the one or more speech qualities of the user.

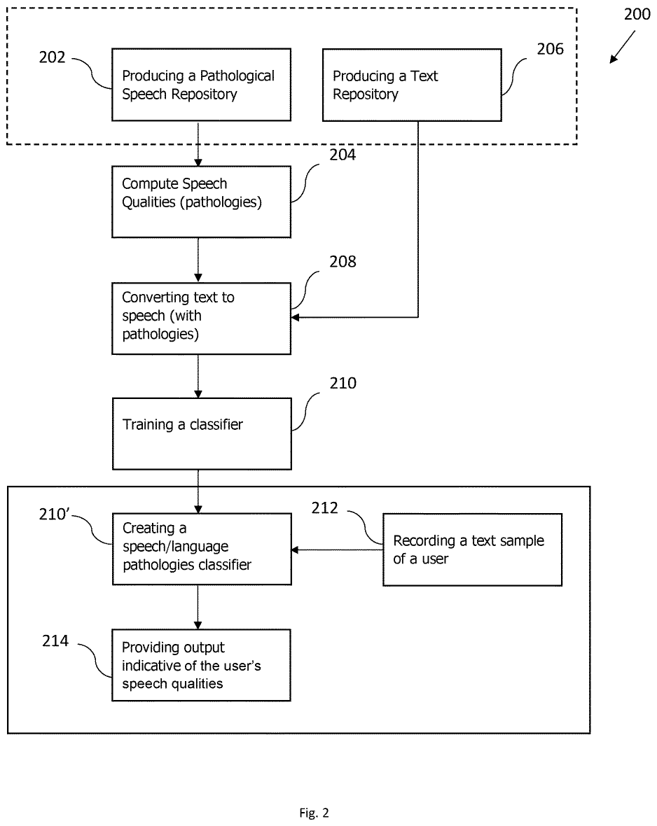

Reference is now made FIG. 2, which schematically depicts a flowchart 200 of a method for treating/diagnosing a speech/language related pathology, according to some embodiments. The method includes the following steps:

Step 202—producing (e.g., generating by a computer and digitally stored) a pathological speech repository of pathological speech samples of various impairments (for example, but not limited to, stuttering, pronunciation pathologies, phonation pathologies, voice related pathologies, Parkinson related speech impairment, impaired articulation language impairments, etc.).

Step 204—based on data receive from the pathological speech repository, computing speech qualities (SQs), for example, parameters, features and/or attributes of speech impairments that will be needed to drive the Text-To-Speech (TTS) synthesis.

Step 206—producing a text repository, which includes a collection of text passages. Step 206 may be conducted before, after or simultaneously with steps 202/204.

Step 208—converting the text passages (formed in step 206) to speech, while introducing to the converted speech, the speech pathologies computed in step 204, thus creating multiple synthetic impaired speech segments.

Step 210—training a classifier with the multiple synthetic impaired speech segments produced in step 208 (for example, implementing machine learning software) and thus creating a speech/language pathologies classifier (step 210′).

Step 212—Recording a speech (spoken text) sample of a user (the user may read any text presented to him/her, whether from the repository, from other sources or speak spontaneously) and introducing it to the speech/language pathologies classifier. The result output is indicative of the user's speech qualities (Step 214).

In the description and claims of the application, each of the words “comprise” “include” and “have”, and forms thereof, are not necessarily limited to members in a list with which the words may be associated.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method of creating a speech/language pathologies classifier, the method comprising:
producing a pathological speech repository of pathological speech samples of multiple impairments;
computing speech qualities/pathologies, based on data receive from the pathological speech repository;
producing a text repository, the text repository comprises multiple known text passages;
converting each one of a selection of the text passages from the multiple known text passages, to a speech segment, while introducing to the speech segment one or more of the computed speech pathologies, thereby creating multiple synthetic impaired speech segments; and
training a classifier with the multiple synthetic impaired speech segments thereby creating a speech/language pathologies classifier.

2. A method for personalized speech therapy, the method comprising:
recording an actual speech sample provided by a user; and
utilizing the speech/language pathologies classifier provided in claim 1, computing one or more output signals indicative of one or more speech qualities of the user.

3. The method of claim 1, wherein training the classifier comprises implementing a machine learning software.

4. The method of claim 2, wherein the output signal further comprises one or more assigned speech quality scores.

5. The method of claim 1, wherein the one or more speech qualities comprises speech intelligibility, fluency, vocabulary, accent, emotion, pronunciation, jitter, shimmer, duration, intonation, tone, rhythm, or any combination thereof.

6. The method of claim 2, wherein the output signal further comprises one or more assigned speech intelligibility scores.

7. The method of claim 2, further comprising providing a feedback signal to the user and/or to a caregiver.

8. The method of claim 1, wherein the step of producing the pathological speech repository of pathological speech samples of multiple impairments comprises recording of speech samples from human subjects.

9. The method of claim 2, wherein the step of recording the actual speech sample is provided by the user in response to a content-containing stimulus.

10. The method of claim 2, wherein the content-containing stimulus comprises a text section, a picture, an image, a video clip, a vocal section or any combination thereof, presented to the subject.

11. (canceled)

12. A system for personalized speech therapy, the system comprising:
a recorder configured to record an actual speech sample of a user; and
a processor comprising:
a speech qualities module configured to compute speech qualities/pathologies based on data receive from a pathological speech repository of pathological speech samples of multiple impairments;
a Text to Speech module configured to convert text passages, obtained from a text repository comprising multiple known text passages, to a speech segments, while introducing to the speech segments one or more of the computed speech pathologies, thereby creating multiple synthetic impaired speech segments; and
a speech/language pathologies classifier configured to receive the multiple synthetic impaired speech segments and the recorded speech sample of the user and to produce an output signal indicative of one or more speech qualities of the user.

13. The system of claim 12, further comprising a recorder configured to record a text sample of a user and to introduce it to the speech/language pathologies classifier.

14. The system of claim 12, further comprising a display configured to present the one or more speech qualities of the user.

15. (canceled)

16. A method of training a subject suffering from a speech pathology, the method comprising:
recording a user's speech section;
utilizing voice analysis algorithms, analyzing the user's speech section to identify at least one speech impairment;
modifying the identified speech impairment to produce a synthetic speech section comprising a modified speech impairment; and
playing to the user the synthetic speech section having the modified speech impairment, thereby providing a feedback to the user regarding the speech thereof.

17. The method of claim 16, wherein the synthetic speech section is produced by using or mimicking the user's own voice or one or more voice qualities of the user.

18. The method of claim 16, wherein modifying the speech impairment comprises removing the speech impairment.

19. The method of claim 16, wherein modifying the speech impairment comprises adjusting the level of the speech impairment.

20. The method of claim 16, wherein modifying the speech impairment comprises shifting the time and/or frequency of the impairment.

21. The method of claim 16, wherein playing to the user the synthetic speech section comprises playing the section in a time delay (Delayed Auditory Feedback).

22. The method of claim 16, further comprising computing a speech quality score based on a comparison between the user's recorded speech and a template (normal) speech section.

23. (canceled)

24. (canceled)

25. (canceled)

26. (canceled)
